(19) United States
(12) Patent Application Publication
(10) Pub. No.: US 2018/0139993 A1
(43) Pub. Date: May 24, 2018

(54) FERMENTATIVE METHOD FOR BLEACHING BIOMASS OF CHLORELLA PROTOTHECOIDES

(71) Applicant: Roquette Freres, Lestrem (FR)

(72) Inventors: Marie Le Ruyet, Lille (FR); Laurent Segueilha, Marquette Lez Lille (FR); Sylvain Delaroche, Longuenesse (FR)

(73) Assignee: Roquette Freres, Lestrem (FR)

(21) Appl. No.: 15/575,156

(22) PCT Filed: May 18, 2016
(86) PCT No.: PCT/FR2016/051163

§ 371 Date: Nov. 17, 2017

(30) Foreign Application Priority Data
May 19, 2015 (FR) 1554442

(51) Int. Cl. A23L 17/60; A23J 3/20; C12N 1/12

(52) U.S. Cl. CPC A23L 17/60; C12N 1/12; A23J 3/20

(57) ABSTRACT

The invention relates to a method for bleaching a biomass of Chlorella protothecoides microalgae that is rich in protein, said biomass being produced by fermentation, characterised in that it comprises: choosing to produce the microalga biomass under heterotrophic conditions and under continuous fermentation; and varying the colouring of said biomass by monitoring the ratio of the μ/μmax growth rates.

The present invention relates to a fermentative process for bleaching biomass of microalgae, more particularly of the Chlorella genus, even more particularly of the species

Chlorella protothecoides.

Macroalgae and microalgae have a specific richness which remains largely unexplored. Their utilization for dietary, chemical or bioenergy purposes is still highly marginal. Nonetheless, they contain components of great value.

Indeed, microalgae are sources of vitamins, lipids, proteins, sugars, pigments and antioxidants.

Algae and microalgae are thus of interest to the industrial sector, where they are used for manufacturing food supplements, functional foods, cosmetics and medicaments, or for aquaculture.

The use of biomasses of microalgae (and principally the proteins thereof) as food is being increasingly considered in the search for alternative sources to meet the increasing global demand for animal proteins (as reported aby the FAO).

Moreover, the European Union has been suffering from a structural deficit in plant proteins for years now, which has amounted in recent years to more than 20 million tons of soy equivalent, currently imported from South America.

The mass production of certain protein-rich microalgae is thus envisioned as a possible way to reduce this “protein deficit”.

Extensive analyses and nutritional studies have shown that these algal proteins are equivalent to conventional plant proteins, or even are of superior quality.

Nonetheless, due to the high production costs and technical difficulties in incorporating the material derived from microalgae into organoleptically acceptable food preparations, the widespread distribution of microalgal proteins is still in its infancy.

Indeed, while algal powders for example produced with algae photosynthetically cultured in exterior ponds or using photobioreactors are commercially available, they have a dark green color (associated with chlorophyll) and a strong, unpleasant taste.

Even formulated in food products or as nutritional supplements, these algal powders always give this visually unattractive green color to the food product or to the nutritional supplement and have an unpleasant fishy taste or the taste of seaweed.

There is therefore still an unsatisfied need for compositions of biomass of microalgae of the Chlorella genus of suitable organoleptic quality, allowing the use thereof in more numerous and diversified food products.

A first solution proposed has been to select Chlorella variants which have a low content or absence of chlorophyll pigments. Thus, Prototheca is conventionally described as a colorless Chlorella.

A second solution consists in irradiating with X-rays or UV-rays, or treating with chemical agents (NTG) or physical agents (heat treatment), a parental strain in order to select depigmented mutants.

The culturing of these mutants is preferentially carried out under heterotrophic conditions (in the dark and in the presence of a nutritive carbon-based source, such as glucose) in order to maintain a selection pressure.

However, these technical solutions do not automatically guarantee the stability of these depigmented variants, or the preservation of the quality, richness and/or diversity of the other components of interest of the biomass.

There is therefore still an unsatisfied need for compositions of biomass of microalgae of the Chlorella genus of suitable organoleptic quality, still having the same richness in components of interest, such as proteins, allowing the use thereof in more numerous and diversified food products.

### SUMMARY OF THE INVENTION

The present invention relates to a process for bleaching a biomass of Chlorella microalgae rich in proteins, characterized in that:
the microalgal biomass is produced under heterotrophic conditions and under continuous fermentation conditions, and
the coloring of said biomass is decreased by increasing the μ/μmax growth rate ratio.

Preferably, the microalgal biomass is bleached by parameterizing the conducting of continuous fermentation in such a way that the μ/μmax growth rate ratio is greater than or equal to 0.90, preferably greater than or equal to 0.95.

Preferably, the continuous fermentation is carried out in a chemostat.

Preferably, the microalgal biomass rich in proteins has a protein content of more than 50% expressed in N 6.25. Preferably, the Chlorella microalga is Chlorella protothecoides.

### DETAILED PRESENTATION OF THE INVENTION

The present invention thus relates to a process for producing a biomass of Chlorella microalgae rich in proteins by controlling the coloring of the biomass, characterized in that:
it is chosen to produce the microalgal biomass under heterotrophic conditions and under continuous fermentation conditions,
the coloring of said biomass is varied by controlling the μ/μmax growth rate ratio.

For the purposes of the present invention, the term “biomass rich in proteins” is intended to mean a biomass which has a protein content of more than 50%, preferably of more than 53%, even more preferably of more than 55%, expressed in N 6.25.

Preferably, the microalgae of the Chlorella genus are chosen from the group consisting of Chlorella vulgaris, Chlorella sorokiniana and Chlorella protothecoides, and are more particularly Chlorella protothecoides. In one particular embodiment, the strain is Chlorella protothecoides (strain UTEX 250—The Culture Collection of Algae at the University of Texas at Austin—USA). In another particular embodiment, the strain is Chlorella sorokiniana (strain UTEX 1663—The Culture Collection of Algae at the University of Texas at Austin—USA).

It has been noted entirely surprisingly by the inventors that the coloring is inversely correlated to the μ/μmax growth rate ratio: the coloring decreases when the μ/μmax growth ratio increases and it increases when the μ/μmax growth ratio decreases.

The biomass of Chlorella rich in proteins of the invention is known for its marked green color, linked to its natural content of chlorophyll.

For the purposes of the invention, “the bleaching of the biomass” is intended to mean the changing of the basic green color to a yellow color, going through all the shades from green to yellow.

The measuring of the green or yellow color can be carried out using any colorimetric model known as such by those skilled in the art.

The HSL (acronym for Hue, Saturation, Lightness) model, which is based on the sensation of human perception, hence its name of perceptual model, may be chosen.

The three criteria which characterize the HSL are the hue, the saturation and, finally, the lightness. The saturation reflects well the intuitive notion of coloring, since it goes from vivid colors to gray. The lightness is measured between black (no light or value 0) and white (maximum light or value 1).

The major advantage of the HSL model is that it clearly separates the lightness component from the chromatic components. Hues and saturation are on one and the same plane in conformity with the colored sensation of the eye and the lightness, for its part, is placed on a perpendicular axis.

It is also possible to use the L, a, b system established by the Commission Internationale de I'Eclairage [International Commission on Lumination], which consists of a three-dimensional Cartesian reference frame (L, a, b) wherein the “L” axis represents the clarity, the “a” axis represents a shade of color between red and green, and the “b” axis represents a shade of color between yellow and blue.

By way of illustration, the tables below present the measurements carried out according to the HSL model and according to the L, a, b system for the two colors that are the greens and yellows at the end of the color sequence conventionally measured for the biomasses according to the invention (measurements carried out in triplicate).

TABLE 1

| | Green | | | Yellow | | |
|---|---|---|---|---|---|---|
| | H | S | L | H | S | L |
| | 49 | 237 | 68 | 38 | 231 | 188 |
| | 51 | 223 | 68 | 39 | 227 | 187 |
| | 51 | 217 | 68 | 38 | 235 | 192 |
| Standard | 1 | 10 | 0 | 1 | 4 | 3 |
| deviation | | | | | | |
| Mean | 50 | 226 | 68 | 38 | 231 | 189 |
| | Green | | | Yellow | | |
| | L | a | b | L | a | b |
| | 49 | −26 | 52 | 97 | −9 | 52 |
| | 55 | −26 | 56 | 96 | −9 | 48 |
| | 53 | −26 | 54 | 97 | −9 | 48 |
| Standard | 3 | 0 | 2 | 1 | 0 | 2 |
| deviation | | | | | | |
| Mean | 52 | −26 | 54 | 97 | −9 | 49 |

As will be exemplified hereinafter, the applicant company has chosen to express the color of the biomass produced using the HSL model, the L, a, b model being suitable instead for measuring the colorimetric variations of powders that are white to yellow (measurement of the balance of yellows, “b” axis or “Yellow index”).

As regards the continuous fermentation process, it should be understood here to mean more particularly carrying out fermentation with addition of sterile medium and drawing off performed within one and the same fermentation cycle.

However, the recourse to this continuous fermentation process is not intended here to increase the biomass productivity, but to control the color of the biomass produced.

The applicant company has in fact found that, surprisingly and unexpectedly, it is possible to vary the coloring of said biomass by controlling the μ/μmax growth rate ratio.

To illustrate this result, the applicant company recommends carrying out this continuous fermentation in a chemostat: the fresh medium is then provided at a constant flow rate (F), and then removed from the fermenter at the same flow rate, thus maintaining a constant culture volume (V).

In the stable state, the growth rate “μ” of the culture is equal to the dilution rate “D”, and is defined by the relationship D=F/V.

The concentration of biomass and other parameters stabilize at values that depend on the dynamics of the fermentation.

Fundamentally, the chemostat culturing allows those skilled in the art to control the growth rate (μ) at a value below a maximum value called the maximum growth rate (μmax).

This is in contrast to batch-mode cultures, wherein the concentration of the biomass and the environmental conditions (in terms of pH, nutrients concentration, etc.) change significantly over the course of the (limited) duration of the fermentation, and those skilled in the art have no control over the growth rate.

The factor which determines the growth rate of a population of cells in a chemostat is the dilution rate, that is to say the feed flow rate of the limiting nutritive element

In the process of the invention, it is in this case glucose.

In a chemostat operating at a low dilution rate, the glucose is present at very low concentrations in the stationary state.

Consequently, most of the nutritive elements are converted in cells and the concentration of the biomass is high (close to the cellular concentration of an equivalent batch culture at the end of the exponential phase).

When the dilution rate increases, the glucose availability increases; however, the speed of elimination of the cells from the fermenter is also higher and the concentration of the biomass thus falls.

At values of D close to μmax, the nutrient limitation disappears entirely because there are too few cells in the culture which use the available nutrients.

The cells remaining in the fermenter grow at their maximum growth rate since the same limiting nutrient is present in excess.

Finally, at D greater than μmax, a state of equilibrium can no longer be maintained and the number of cells in the fermenters begins to decrease since the speed at which new cells are produced is insufficient to avoid dilution by the addition of fresh medium, which results in washing of the cells from the fermenter.

These data make it possible to explain the reason why, while fermentation in a chemostat is recommended for increasing productivity at zero residual glucose, those skilled in the art will choose to run the chemostat in such a way that D is less than μmax in order to obtain the highest concentration of biomass.

For its part, the applicant company has found that, the more p is increased toward μmax, the more the productivity increases but also the more the biomass produced is bleached. As will be exemplified hereinafter, the parameters used in this continuous fermentation are, depending on the conditions exemplified hereinafter, the following:
pH=5.2 adjusted with 10% (v/v) aqueous ammonia,
pO₂=30% adjusted in cascade on the shaking,
aeration: 1 vvm (1.5 l/min).

At equilibrium:
feed flow rate=output flow rate=0.120 l/h to 0.180 l/h
dilution rate=μ=0.02 h⁻¹ to 0.12 h⁻¹
biomass concentration=50 g/l to 100 g/l
glucose concentration=100 g/l to 200 g/L.

In one example, the chemostat is equilibrated over the course of 5 renewals (62.5 h if D=0.08 h⁻¹). Samples are taken after the 5 renewals and more than 7 h subsequently, in order to verify the state of equilibrium of the chemostat.

It is thus found that the green-colored basic Chlorella protothecoides biomass will be gradually bleached toward the yellow color if μ is increased from 0.08 h⁻¹ to 0.1 h⁻¹.

Conversely, if μ is reduced from 0.08 h⁻¹ to 0.06 h⁻¹, the coloring of the biomass is strengthened toward the green tones.

Moreover, and this is what is essential in the process in accordance with the invention, the conducting of the fermentation in this way in a chemostat does not modify the composition of the biomass produced.

As will be exemplified hereinafter, whether the growth rate is weak (0.06 h⁻¹) or strong (0.1 h⁻¹), no significant effect on the composition of the Chlorella protothecoides biomass produced has been observed.

The main differences are instead in terms of the production speeds which, overall, are improved with a strong p.

By way of example, for Chlorella protothecoides, the best results obtained make it possible to achieve a productivity of 9.5 g of biomass/l/h while at the same time preserving a protein content, expressed in % of N6.25, of more than 55%.

Once again, it is more surprising to note that the variation in μ has a considerable impact on the color of the biomass produced.

Without being bound by any theory, the applicant company considers, with regard to the results obtained (on the basis of the representation by the HSL model of the hue H of the biomass as a function of the lightness L for each measurement), that:
the variations in the hue could correspond to the gradual decrease in the production of chlorophyll which masks the other pigments (carotenoids),
the lightness, for its part, could correspond further to the decrease in the overall concentration of pigments.

As will be exemplified hereinafter, the level of color considered to be “correct” is that for which (for a hue range of between 30 and 60 and a lightness range of between 50 and 230):
the hue value is less than 40,
the lightness value is greater than 135, preferably greater than 150 and even more preferably greater than or equal to 170.

The invention will be understood more clearly from the following examples which are intended to be illustrative and nonlimiting.

### DESCRIPTION OF THE FIGURES

FIG. 1: Change in the O₂ consumed and in the CO₂ produced as a function of the imposed dilution rate.

FIG. 2: Change in the absorbance, in the glucose and in the biomass content as a function of the imposed dilution rate.

FIG. 3: Change in the hue and in the lightness as a function of the imposed dilution rate.

FIG. 4: Change in the hue as a function of the μ/μmax growth rate ratio.

FIG. 5: Change in the lightness as a function of the μ/μmax growth rate ratio.

FIG. 6: Change in the hue and in the lightness as a function of the D/Dmax dilution rate ratio.

### EXAMPLES

### Example 1

### Determination of the Value of μMax and Change in the Color of the Biomass as a Function of the μ/μMax Ratio in an Accelerostat

The strain used is Chlorella protothecoides UTEX 250 (The Culture Collection of Algae at the University of Texas at Austin—USA).

The fermentation is carried out in an accelerostat, which is a variant of the chemostat in which the fermenter never comes into stationary dynamic equilibrium. In fact, the D is increased in a linear and gradual manner starting from a low value.

The advantage of this technique is the study of a wide range of dilution rates and also the rapid and precise access to the μmax of the strain under the conditions implemented.

The fermentation conditions are the following, for the production of 100 g/l of biomass:

Feed Medium
Glucose: 200 g/l
(NH₄)₂SO₄: 1 g/l
NH₄H₂PO₄: 8 g/l
MgSO₄.7H₂O: 4.8 g/l
FeSO₄.7H₂O: 0.020 g/l
CaCl₂.7H₂O: 0.050 g/l
ZnSO₄.7H₂O: 0.025 g/l
MnSO₄.1H₂O: 0.020 g/l
CuSO₄.5H₂O: 0.001 g/l
Thiamine.HCl: 0.020 g/l
Biotin: 0.001 g/l
Pyridoxine: 0.010 g/l

Conducting of Fermentation:
Brought to pH 5.2 with 10% (w/v) NH₄OH
Incubation temperature: 28° C.
Inoculum: 0.5 l of an Erlenmeyer flask of preculture having an OD_{750 nm}=15 in a fermenter of 2 l (1.5 l medium apart from shaking, before addition)
Pulse of Sigma S208 antifoam 50% ethanol (v/v) at a rate of one pulse of 1 second per hour
Shaking: 300 rpm at the start
Aeration: 1.5 l/min of air
pO₂Regulation: 30%

The analysis of the gases (O₂/CO₂), the absorbance at 750 nm, the glucose concentration and the weight of cells are the parameters measured in order to determine the value of the μmax.

FIG. 1 presents the change in the O₂ consumed and in the CO₂ produced as a function of the imposed dilution rate.

It appears that the O₂ and the CO₂ change gradually up to 0.05 h⁻¹, and then the curves abruptly invert.

In parallel to the gas measurements, the change in absorbance, in glucose and in biomass content are monitored kinetically.

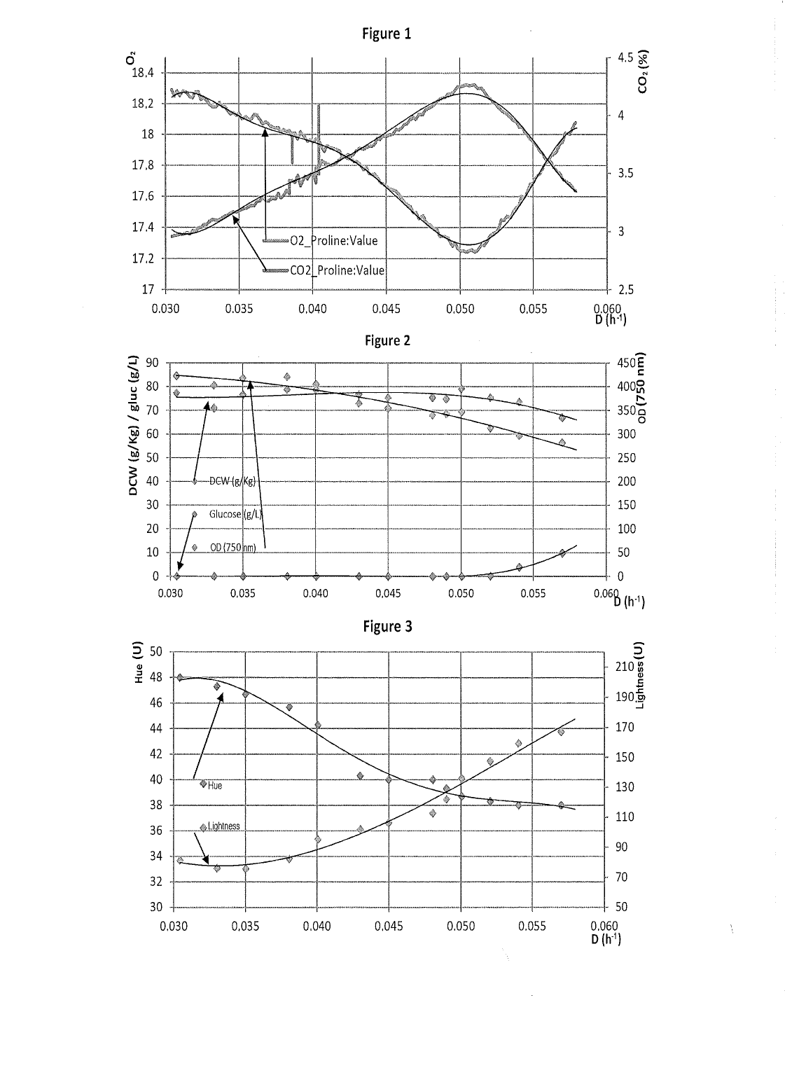

FIG. 2 presents the results obtained.

Once again, starting from 0.05 h⁻¹, there is:
a beginning of accumulation of residual glucose,
an inflection of the curve of biomass (initially stable at 75 g/l of dry cells),

which indicates that the chemostat is beginning to enter the leaching phase, that is to say the imposed μ becomes higher than the μmax of the strain under the conditions imposed.

The system thus eliminates more quickly the cells that it does not reproduce, thereby causing the cell load in the reactor to decrease, the O₂ not consumed to increase again and the CO₂ produced to decrease.

The decrease in the absorbance also reflects the decrease in the biomass content starting from a μ of 0.05 h⁻¹.

It results from all these analyses that the Dmax=μmax=0.05 h⁻¹ under the chosen fermentation conditions.

The measurements of color of the biomass produced are carried out according to the HSL model.

FIG. 3 presents the change in the color (hue and lightness) as a function of the dilution rates.

The change in the hue and lightness curves is directly dependent on the μmax.

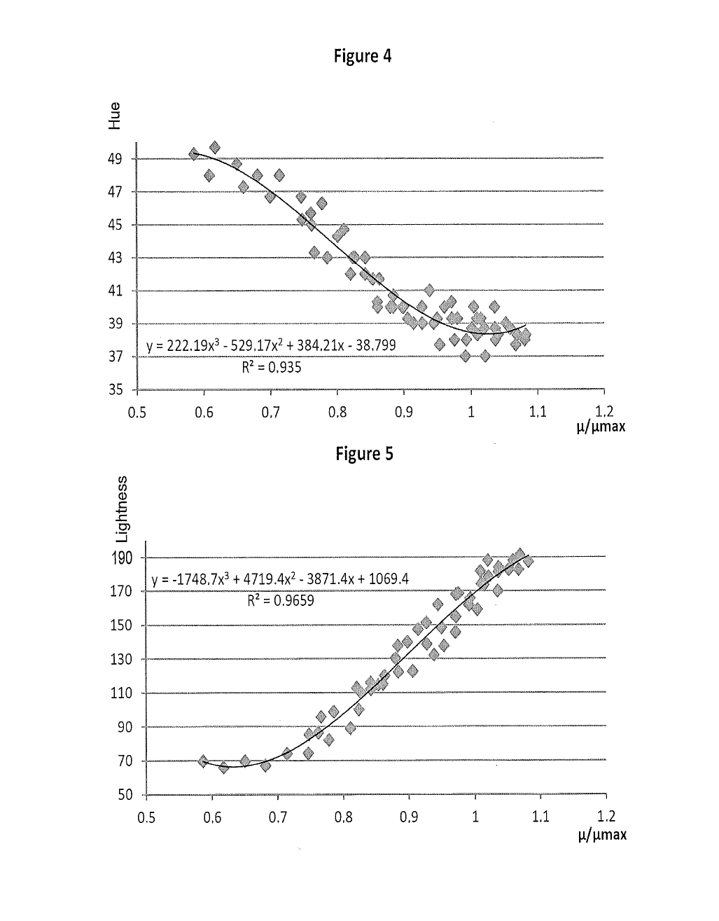

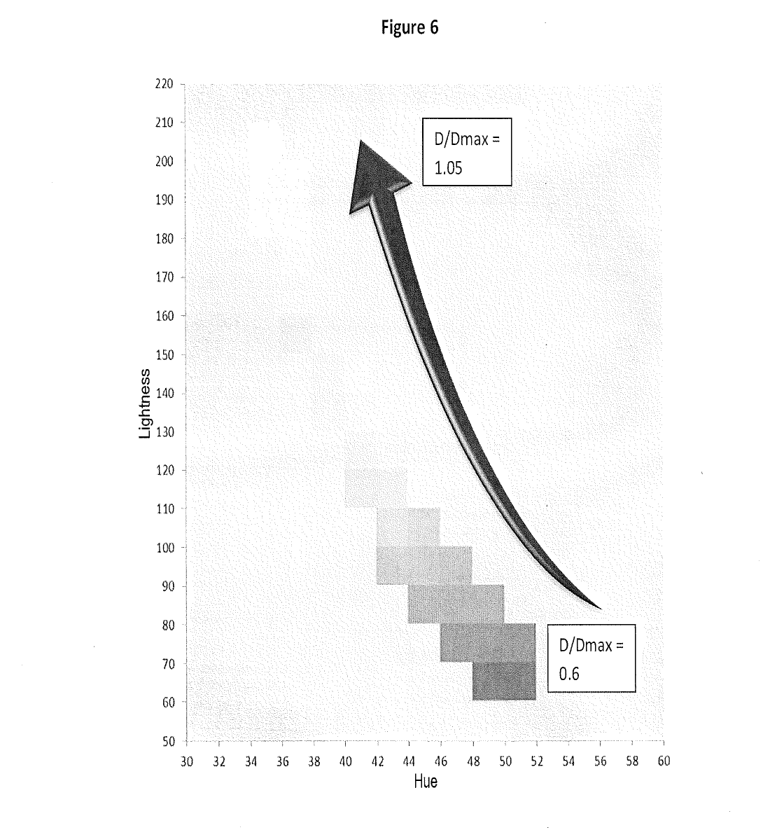

Three representations in graph form make it possible to illustrate the change in the color as a function of the μ/μmax ratio:
change in the hue as a function of the μ/μmax ratio (FIG. 4),
change in the lightness as a function of the μ/μmax ratio (FIG. 5).

It is deduced therefrom that:
the color changes very strongly throughout the time of the accelerostat (that is to say as a function of the value of D),
the most yellow and bleached biomass is obtained when p tends toward μmax, under the chosen operating conditions.

### Example 2

### Change in the Color of the Biomass as a Function of the Dilution Rate (and Thus the Value of μ) of Points of Equilibrium in a Chemostat

Chemostats are run under the conditions identical to example 1, but with a medium that is two times less concentrated such that the biomass concentration at equilibrium is 50 g/l. The μmax on this medium is 0.104 h⁻¹.

The following measurements were carried out at various dilution rates once the equilibrium was obtained (5 renewals). They confirmed the effect of the μ/μmax ratio on the coloring:

TABLE 2

| Tests | 1 | 2 | 3 | 4 |
|---|---|---|---|---|
| D (h⁻¹) | 0.102 | 0.028 | 0.088 | 0.064 |
| μ/μmax | 0.98 | 0.27 | 0.85 | 0.61 |
| Hue | 36 | 52 | 47 | 48 |
| Lightness | 171 | 68 | 86 | 81 |

Conversely, the composition of the biomass is not significantly modified, as shown by the contents of proteins (N 6.25 and Total Amino Acids), total fatty acids and total sugars which are given below.

TABLE 3

| Tests | 1 | 2 | 3 | 4 |
|---|---|---|---|---|
| N 6.25 (% dry) | 60.0 | 56.1 | 54.2 | 57.2 |
| Total amino | 37.6 | 40.6 | 39.5 | 40.6 |
| acids (% dry) | | | | |
| Total fatty acids | 8.4 | 7.0 | 7.2 | 7.6 |
| (% dry) | | | | |
| Total sugars (% | 31.2 | 24.8 | 30.1 | 26.2 |
| dry) | | | | |

1-6. (canceled)

7. A process for bleaching a biomass of Chlorella microalgae rich in proteins, comprising:
continuously producing a biomass of Chlorella microalgae rich in proteins under heterotrophic conditions at a growth rateμ and having a maximum growth rate of μmax, and bleaching said biomass to decrease color by increasing a ratio of μ/μmax.

8. The process according to claim 7, wherein said bleaching is parameterized by conducting continuous fermentation such said ratio is greater than or equal to 0.90.

9. The process according to claim 7, wherein ratio is greater than or equal to 0.95.

10. The process according to claim 7, wherein said continuous fermentation is carried out in a chemostat.

11. The process according to claim 7, wherein said microalgal biomass rich in proteins has a protein content of more than 50%, expressed in N 6.25.

12. The process according to claim 7, wherein the Chlorella microalga is Chlorella protothecoides.

13. A process of varying a biomass color produced from Chlorella microalgae rich in proteins, comprising:
producing a biomass of Chlorella microalgae rich in proteins under heterotrophic conditions, adjusting a growth rate μ of said biomass and varying said biomass color by increasing a ratio of said growth rate μ relative to a growth rate max to a value of at least 0.9 to decrease the biomass color.

14. The process according to claim 7, wherein the biomass comprises a hue range of between 30 and 60 and a lightness range of between 50 and 230.

15. The process according to claim 13, wherein the biomass comprises a hue range of between 30 and 60 and a lightness range of between 50 and 230.

16. A process of varying a biomass color produced from Chlorella microalgae rich in proteins, comprising:
operating a chemostat containing a biomass of Chlorella microalgae rich in proteins under heterotrophic conditions having a first color, and varying said biomass color by increasing a ratio of a growth rate μ of said biomass relative to a growth rate μmax, where said ratio is a value of at least 0.9 to decrease the biomass color.

17. The process according to claim 7, wherein the Chlorella is one of Chlorella vulgaris, Chlorella sorokiniana and Chlorella protothecoides.

18. The process according to claim 7, wherein the Chlorella is one of Chlorella vulgaris, Chlorella sorokiniana and Chlorella protothecoides.

19. The process according to claim 7, wherein a protein content, expressed in % of N6.25, of more than 55% is preserved.

20. The process according to claim 13, wherein the protein content, expressed in % of N6.25, of more than 55% is preserved.

21. The process according to claim 16, wherein the protein content, expressed in % of N6.25, of more than 55% is preserved.
